(19) United States
(12) Patent Application Publication
(10) Pub. No.: US 2005/0181042 A1
(43) Pub. Date: Aug. 18, 2005

(54) Composition for oral or rectal administration

(76) Inventor: Bengt Herslof, Stockholm (SE)

Correspondence Address: DICKSTEIN SHAPIRO MORIN & OSHINSKY LLP, 1177 AVENUE OF THE AMERICAS (6TH AVENUE), 41 ST FL., NEW YORK, NY, 10036-2714, US

(21) Appl. No.: 10/504,490

(22) PCT Filed: Feb. 14, 2003
(86) PCT No.: PCT/SE03/00251

(30) Foreign Application Priority Data
Feb. 15, 2002 (SE) 0200475-2

(51) Int. Cl. A61K 9/20; A61K 31/739

(52) U.S. Cl. 424/464; 514/54

(57) ABSTRACT

A solid pharmaceutical or food supplement tablet or suppository composition has a melting point of 25° C. or higher and comprises a continuous lipid component comprising one or more polar lipids, one or more non-polar lipids, optionally one or several of water and mono- to trivalent alcohol in an amount of up to 15% by weight of the composition, and one or more agents selected from pharmacologically active agent and food supplement agent. Also disclosed is a corresponding tablet and a corresponding suppository, processes for production of the composition and the tablet and the suppository, and a method of preventing or treating conditions amenable to preventive or therapeutic treatment by administration of the tablet or suppository.

### FIELD OF THE INVENTION

The present invention relates to a pharmaceutical tablet and suppository composition for oral or rectal administration based on lipid carrier materials and to methods for its manufacture and administration.

### BACKGROUND OF THE INVENTION

From a standpoint of patient convenience and production technology the most attractive pharmaceutical form for oral administration of pharmaceutical agents is the tablet but in some cases also rectal administration by a suppository may be advantageous. However far from all pharmaceutical agents are easily formulated as tablets or suppositories. This is true, in particular, to many active principles which are not easily absorbed from the gastrointestinal tract and require, for optimal absorption to be delivered in pharmaceutical carriers comprising lipids which cannot be compounded as tablets or suppositories. Hard or soft shell capsules have to be used instead. However the preferred capsule material gelatin often is not sufficiently inert towards pharmaceutical excipients of this sort and limits the shelf life of the capsule preparation or requires the use of hard gelatin capsules. Hard gelatin capsules are however particularly inconvenient to swallow. In recent years there has also been some concern among consumers in regard of gelatin obtained from animal sources.

On the other hand oral administration of pharmaceutical agents in lipid based carriers contained in capsules undeniably has resulted in improved drug performance in terms of bioavailability. Examples include such compounds as cyclosporin and saquinavir, marketed under the name of Sandimmun Neoral®, Novartis and Invirase®, Roche respectively. Such lipid based carriers are either oily liquids, such as microemulsions, or dispersions, such as emulsions or liposomal preparations, which cannot be easily incorporated into tablets.

Numerous reports describe the use of lipids as tablet excipients in combination with non-lipid constituents. A background of the state of the art in regard of tablet formulations is given in “Modern Pharmaceutics” (Editors G. Banker and C. Rhodes, Marcel Dekker Inc., New York 1996, chapter 10, pp 333-394). Most tablets are manufactured by means of powder compression. The pharmaceutical agent(s) is (are) mixed with the excipients to produce a free-flowing powder. Among commonly used excipients are several that can be classified as lipids, for example glycerol triacetate, glycerol behenate, glycerol palmitostearate, zink stearate, magnesium stearate, calcium stearate, stearic acid, hydrogenated vegetable oils, and waxes. Other lipophilic ingredients include paraffins and light mineral oils. Also synthetic lipophilic and amphiphilic ingredients are used, such as polyethylene glycols (PEG), polyoxyethylene monostearates, sodium lauryl sulphate, and sucrose monolaurate.

Most of the aforementioned lipid ingredients act as soluble or insoluble lubricants. They are combined with other types of ingredients, such as fillers (e.g., lactose and starch), binders (e.g., starch mucilage), and disintegrants (e.g., microcrystalline cellulose and cross-linked polyvinylpyrrolidone). Besides their lubricating function lipid ingredients have been used in controlled release formulations.

WO 95/20945 discloses a lipophilic carrier preparation in form of an oily liquid or dispersion having a continuous lipid phase, comprising a non-polar lipid in combination with a polar lipid material, and optionally a polar solvent, polar lipid material being a galactolipid material consisting of at least 50% digalactosyldiacylglycerols, the remainder being other polar lipids.

WO 92/05771 discloses a lipid particle forming matrix containing bioactive material(s) comprising at least two lipid components, one being non-polar and the other amphiphatic and polar. When brought in contact with an aqueous solvent the matrix spontaneously forms discrete lipid particles. The amphiphatic and polar lipid matrix components are said to be bilayer forming and are chosen from phospholipids such as phosphatidylcholine; the non-polar lipids are mono-, di- or triglycerides.

### OBJECTS OF THE INVENTION

It is an object of the invention to provide a solid pharmaceutical or food supplement tablet or suppository composition which exploits the advantageous properties of lipids as pharmaceutical carriers in regard of gastro-instestinal uptake and/or sustained release and/or convenience and/or economy.

It is another object of the invention to provide a corresponding carrier composition for incorporation of pharmacologically active or food supplement agents.

It is a further object of the invention to provide processes for making the aforementioned compositions and for incorporating a pharmacologically active agent or food supplement into said carrier composition.

Further objects of the invention will be evident from the following short description of the invention, the description of preferred embodiments, and the appended claims.

### SHORT DESCRIPTION OF THE INVENTION

According to the present invention is disclosed a solid pharmaceutical or food supplement tablet composition which has a melting point of from 25° C. to 50° C. or more, preferably from 30° C. to 45° C., more preferred from 33° C. to 42° C., comprising a continuous lipid phase comprising, preferably consisting of, a polar lipid component, a non-polar lipid component, and a pharmacologically active agent. The polar lipid component consists of one or more polar lipids. The non-polar component consists of one or more non-polar lipids. The one or more polar lipids are membrane lipids, in particular glycolipids and phospholipids. The one or more non-polar lipids are preferably glycerides, i.e. glycerol esters of fatty acids (mono-, di-, and triglycerides). All polar and non-polar lipids of the invention can be sourced from foodstuffs or food grade material. The polar lipids of the invention are amphiphilic with headgroups such as galactose or phosphate esters. The polar lipid component of the invention is combined with the non-polar lipid component in various proportions to allow the controlled incorporation of pharmaceutical including food supplement agents. It is believed that the incorporation mechanism is based on interactions of the polar headgroups and the lipophilic chains of the non-polar component with the compound to be incorporated. Pharmacologically (including food supplementing) efficient compositions for a given pharmacologically active agent or mixture of agents can be experimentally determined by varying the ratio of the polar to non-polar component. To a certain extent the pharmacological or food supplemental efficacy is also influenced by the composition of the polar and non-polar component, respectively.

Preferably the polar component of the composition according to the invention comprises or, more preferred, consists of one or several polar lipids of vegetable origin, such as oat kernels or soybeans. Preferably the non-polar lipid component of the composition according to the invention comprises or, more preferred, consists of one or several glycerides of vegetable origin, such as palmkernel oil, coconut oil, palm oil and cottonseed oil.

It is particularly preferred for the solid pharmaceutical or food supplement tablet or suppository composition of the invention to comprise lipid material of vegetable origin only.

According to the present invention is also disclosed a solid tablet produced from the aforementioned pharmaceutical or food supplement composition, in particular by compression moulding or casting.

According to the present invention is also disclosed a suppository produced from the aforementioned pharmaceutical composition, in particular by compression moulding or casting.

In the pharmaceutical literature lipid continuous phases are described as oily liquids, which need to be administered as oral liquids or enclosed in hard or soft shell capsules. However, such oily liquids are completely outside of the scope of the present invention. Lipid phases are also known in form of dispersions, i.e. dispersed aqueous solvents. Lipid emulsions and liposome preparations are examples of such dispersions which, by definition, are not lipid continuous phases and therefore do not form part of the present invention.

The polar component of the invention can be described as formed of membrane lipid(s), i.e. the lipid constituents of biological membranes. Membrane lipids contain a polar, hydrophilic, head group and one or more lipophilic hydrocarbon chains. This combination makes the membrane lipid molecules amphipathic and enables them to associate both with water and oils. Such membrane lipids can be classified according to their chemical structure, which is a function of how the polar head group is linked to the lipophilic chains. Sphingolipids (linked by sphingosine) and glycerolipids (linked by glycerol) are the two main groups. Depending on the characteristics of the polar head group sphingolipids and glycerolipids can be further classified as phospholipids, with the head group being a phosphate ester, or as glycolipids, with the head group being a carbohydrate. Depending of the specific nature of the carbohydrate group membrane lipids sometimes are called, for example, galactolipids, which are glycerolipids with galactose in the polar head group. Examples of common membrane lipids are phosphatidylcholine (PC), phosphatidylethanolamine (PE), and digalactosyl-diacylglycerol (DGDG). The membrane lipids can be extracted from, for example, egg yolk (egg lecithin), milk and dairy products, soybeans (soy lecithin), other oil crops, oat kernels, and other cereals and grains. These extracts can be further treated to become, for example, PC from soybeans and galactolipids from oats. Preferred polar lipids are galactolipids, in particular galactolipids from oat kernels (CPL-galactolipid) or from soybeans (soy lecithin or soy-PC). Particularly preferred are partially hydrolysed galactolipids.

Synthetic polar lipids and membrane lipid analogues based on a carbohydrate or phosphate ester moiety are comprised by the polar lipid component of the invention.

The preferred non-polar lipids of the invention are fatty acid esters of glycerol. These esters include mono-, di-, and triglycerides. Edible oils are triglyceride oils, from which mono- and diglycerides can be derived. Other non-polar lipids of the invention include vegetable and animal oils from various sources, synthetic oils, fatty acids, natural and synthetic glycerides, sterol esters, fatty alcohols. Synthetic non-polar lipids and fatty acid analogues are also comprised by the invention. A description of the area of polar and non-polar lipids is given in “Fatty Acid and Lipid Chemistry” (Frank Gunstone, 1996, Blackie Academic & Professional, Chapman & Hall).

The triglyceride may be selected from palmkernel oil or natural oils with similarly, relatively high solid fat content or melting range. Preferred non-polar lipids include palmkernel oil fractions, obtained by commercial fractionation of palmkernel oil into specific mixtures of triglycerides, e.g. palmkernel stearin, based on the combination of mainly lauric, myristic, and palmitic esters of glycerol. Preferred monoglycerides are selected from edible oil derived monoglycerides, in particular medium chain monoglycerides (chain length C₈-C₁₀), derived from coconut oil, and normal chain monoglycerides (chain length C₁₆-C₁₈), derived from most vegetable oils.

According to a preferred aspect of the invention the continuous lipid phase may comprise up to 15% by weight, preferably up to 10% by weight, most preferred up to 5% by weight of water and/or an alcohol, including an alkanediol or -triol, such as ethanol, 1,2-propylene glycol, low molecular weight polyethylene glycol, and glycerol. By definition the continuous lipid phase cannot comprise more water or alcohol than is compatible with its property of being continuous.

According to the invention is also disclosed a pharmaceutical or food supplemental or suppository carrier composition consisting of a continuous lipid phase having a melting point of from 25° C. to 50° C. or more, preferably from 30° C. to 45° C., more preferred from 33° C. to 42° C., comprising, preferably essentially consisting of, a polar lipid component in combination with a non-polar lipid component.

According to the present invention is furthermore disclosed a process for the production of a pharmaceutical or food supplement tablet composition or suppository composition which has a melting point of from 25° C. to 50° C. or more, preferably from 30° C. to 45° C., more preferred from 33° C. to 42° C., comprising a continuous lipid phase comprising, preferably consisting of, a polar lipid component, a non-polar lipid component and a pharmacologically active chemical agent or food supplementing agent, comprising mixing a polar lipid component with a non-polar lipid component at a first temperature at which at lease one of said components is in a liquid state, thereby obtaining a liquid continuous lipid phase, dissolving one or more of said agents in the liquid continuous lipid phase, cooling the solution thus obtained or aliquots thereof to a second temperature at which it solidifies, said second temperature ranging from 25° C. to 50° C. or more, preferably from 30° C. to 45° C., more preferred from 33° C. to 42° C. The cooling may produce a cake if carried out in bulk or a powder if the liquid product is fed to a nozzle, preferably at a temperature slightly above its melting point, and sprayed on, for instance, a cooled metal surface, in particular a polished chromium plated stainless steel surface in form of a band running on rollers. A powderous product may also be obtained by spraying the liquid product into a atmosphere of a temperature below the solidification temperature of the liquid product. The cake may be transformed into powder by, for instance, grinding at a low temperature.

According to a second preferred aspect is disclosed a tablet or suppository of the invention coated with one or several layers of tablet or suppository, respectively, coating excipients, such as to provide the tablet or suppository with an enteric coat and/or a coat physically stabilizing the tablet or suppository at a temperature at or above its melting point, and a corresponding coating process. Particularly preferred is a tablet or suppository of the invention provided with a first or only coat applied by a dry coating process comprising mechanically working a coating powder into the surface of the tablet or suppository at a temperature at which the tablet or suppository is sufficiently soft for the powder particles to adhere and allow them being worked into its surface but not sufficiently soft for substantial deformation, in particular at a temperature from 25° C. to 10° C. below the melting point of the tablet or suppository. One or more additional layers may be added to the thus coated tablet or suppository by routine pharmaceutical coating processes known in the art. The tablet or suppository of the invention may also be built up around an inert nucleus.

A tablet or suppository according to the invention can be produced from the pharmaceutical or food supplement tablet composition of the invention by compressing the aforementioned powderous product or by moulding or any other suitable process. According to a preferred aspect of the invention the moulding is carried out in a mould covered with an anti-adhering agent or layered with an anti-adhering material, such as amorphous silica, cornstarch and sodium lauryl sulphate, and poly(perfluoro-ethylene), respectively.

The pharmaceutical agent or agents of the invention can be of any type suitable for forming a tablet or suppository composition with the pharmaceutical carrier of the invention, with the proviso that the pharmaceutical agent or agents is soluble in the pharmaceutical carrier and is stable at a temperature above 30° C., preferably above 33° C., most preferred above 40° C., for a time sufficient to incorporate it into the pharmaceutical carrier. In this context “stable” means that no more than 5% by weight of the pharmaceutical agent(s), preferably not more than 2% by weight, most preferred not more than 1% by weight, is degraded or lost during the incorporation process. The term “pharmaceutical agent” comprises any substance that prevents, cures or alleviates an aberrant health state, such as a nutritional defect, in particular vitamin deficiency or a deficiency of essential amino acids, and any substance used for diagnostic purposes which is per-orally administrable. The pharmaceutical agent of the invention can be any of analgesics, anti-inflammatory agents, antihelmintics, anti-antiallergic agents, arrhythmic agents, antibacterial agents, anti-coagulants, antidepressants, antidiabetic agents, anti-epileptics, antifungal agents, antigout agents, anti-histamines, antihypertensive agents, antimalarial agents, antimuscarinic agents, antimycobacterial agents, antineoplastic agents, antiprotozoal agents, antithyroid agents, antiviral agents, anxiolytic agents, betaadrenoceptor blocking agents, cardiac inotropic agents, corticosteroids, cough suppressants, diagnostic agents, diuretics, dopaminergics, enzymes, gastro-intestinal agents, hypnotics, hypothalamic hormones, immunological agents, immunosuppressants, lipid regulating agents, mucolytics, muscle relaxants, neuroleptics, nutritional agents, opoid analgesics, parasympathomimetics, pituitary hormones, parathyroid agents, prostaglandins, sedatives, sex hormones, sympathomimetics, thyroid agents, vasodilators, vitamins, and xanthines. The requirements for incorporation of food supplement agents in the tablet of the invention correspond to those of pharmacologically active agents.

By way of examples it was surprisingly found that the solid pharmaceutical or food supplement tablet or suppository composition of the invention not only can incorporate a wide variety of pharmacologically active agents or food supplement agents of vastly differing chemical structures, but also increases its uptake in the gastrointestinal tract and/or prolongs its efficacy. The present invention thus provides a novel way of improving and widening the use of tablet compositions for pharmaceutical use including food supplement use.

In the following the invention will be explained in more detail by the following, non-limiting examples.

### DESCRIPTION OF PREFERRED EMBODIMENTS

Materials. The lipid materials used are listed in Table 1.

If not indicated otherwise all percentages in the description of preferred embodiments are by weight.

TABLE 1
Lipid materials

| Type of | |
|---|---|
| lipid | Trade name and source |
| PL-1 | Galactolipids from oats (CPL-Galactolipid; Lipid |
| | Technologies Provider AB, Karlshamn, Sweden) |
| PL-2 | PC from soybeans (prepared from soy lecithin Epikuron 135 |
| | F; Lucas Meyer GmbH&Co, Hamburg, Germany) |
| MG-1 | Medium chain monoglyceride (Akoline MCM; Karlshamns AB, |
| | Karlshamn Sweden) |
| MG-2 | Monoglycerides from edible oils (Dimodan CP; Danisco, |
| | Copenhagen, Denmark) |
| TG-1 | Palmkernel stearin (fraction of palmkernel oil; |
| | Karlshamns AB, Karlshamn Sweden) |
| TG-2 | Hydrogenated cotton seed oil (Akofine NF; Karlshamns AB, |
| | Karlshamn Sweden) |

### EXAMPLE 1

### Exemplary Preparation of a Tablet by Compression of a Powderous Mixture of Lipids (Method A)

A mixture of the following ingredients (in g) was prepared:

| | |
|---|---|
| Non-polar lipids (hydrogenated triglycerides; Akofine ™) | 18,00 |
| Polar lipid material (galactolipids; CPL-Galactolipid ™) | 2,00 |
| Vitamin B12 | 0,040 |

The powderous ingredients were blended in a dry mixer. Aliquots (0.50 g) of the homogenous powder were compressed to tablets in a manually operated press (Manesty Machines Ltd, Model no D3). It is also possible to prepare a suppository in this manner by using an appropriate press-form.

### EXAMPLE 2

### Exemplary Preparation of a Tablet by Casting Molten Lipid Mixture into a Mould (Method B)

| Ingredients (in g): | |
|---|---|
| Non-polar lipids (fractionated triglycerides; palmkernel | 18,00 |
| stearin) | |
| Polar lipid material (galactolipids; CPL-Galactolipid ™) | 2,00 |
| Vitamin B12 | 0,040 |

The ingredients were blended and the mixture melted by heating to a temperature of 60° C. and stirred at this temperature for 5 hours when all vitamin B12 had dissolved. Aliquots (0.50 g) of the melted phase were cast in a mould covered with hydrogenated triglyceride (Akofine N™) powder. The mould was cooled in a freezer and the tablets recovered. A suppository can be prepared in a corresponding manner by using an appropriate mould.

### EXAMPLE 3

### Preparation of Tablets Containing Vitamin B12, Folic Acid, Retinyl Palmitate or Desmopressin (as Acetate)

Tablets were prepared according to Method A (as described in Example 1) or Method B (as described in Example 2) with several carrier compositions (Table 1) according to the invention. The 17 preparations thus produced and their relative efficacies are listed in Table 2.

The results demonstrate that the proportions and structure of the lipid phase components affect bioavailability. A range from highly improved (by a factor of 5.3) uptake to highly suppressed uptake, i.e. virtually nil, was observed.

TABLE 2
Pharmaceutical/food supplement tablet preparations

| | | Active prin- | Lipids (% by weight) | | | |
|---|---|---|---|---|---|---|
| | | ciple/tablet | | Non-polar lipid | | |
| | | (0.5 g) | Polar | (% by weight) | | Efficacy |
| Prep. | | Vitamin B12 | (% by | Glyceride | Glyceride | (Reference = |
| no. | Method | (mg) | weight) | I | II | 100) |
| 1 | B | 1 | 20 (PL-1) | 5 (MG-1) | 75 (TG-1) | 33 |
| 2 | B | 1 | 20 (PL-1) | 10 (MG-1) | 70 (TG-1) | 74 |
| 3 | B | 1 | 20 (PL-1) | 15 (MG-1) | 65 (TG-1) | 529 |
| 4 | B | 1 | 20 (PL-1) | 20 (MG-1) | 60 (TG-1) | 191 |
| 5 | B | 1 | 20 (PL-1) | 30 (MG-1) | 50 (TG-1) | 100 |
| 6 | B | 1 | 45 (PL-1) | 35 (MG-1) | 20 (TG-1) | 355 |
| 7 | B | 1 | 57 (PL-1) | 43 (MG-1) | 0 | 148 |
| 8 | B | 1 | 10 (PL-1) | 0 | 90 (TG-1) | 108 |
| 9 | A | 1 | 10 (PL-1) | 0 | 90 (TG-2) | 6 |
| 10 | B | 1 | 20 (PL-1) | 15 (MG-2) | 65 (TG-1) | 43 |
| 11 | B | 1 | 20 (PL-2) | 15 (MG-1) | 65 (TG-1) | 71 |
| 12 | B | 1 | 20 (PL-2) | 20 (MG-1) | 60 (TG-1) | 0 |
| | | Folic acid (mg) | | | | |
| 13 | B | 5 | 20 (PL-1) | 10 (MG-1) | 70 (TG-1) | 93 |
| 14 | B | 5 | 20 (PL-1) | 15 (MG-1) | 65 (TG-1) | 117 |
| 15 | B | 5 | 20 (PL-1) | 20 (MG-1) | 60 (TG-1) | 56 |
| 16 | B | 5 | 10 (PL-1) | 0 | 90 (TG-1) | 81 |
| 17 | A | 5 | 10 (PL-1) | 0 | 90 (TG-2) | 1 |
| | | Retinyl palmitate (mg) | | | | |
| 18 | B | 33 (50000 IE) | 10 (PL-1) | 0 | 90 (TG-1) | 115 |
| 19 | A | 33 (50000 IE) | 10 (PL-1) | 0 | 90 (TG-2) | 6 |
| | | Desmopressin* (μg) | | | | |
| 20 | B | 50 | 20 (PL-1) | 15 (MG-1) | 65 (TG-1) | ** |

* As acetate. ** See Example 5

### EXAMPLE 4

### Test of Tablet Preparations in Healthy Human Volunteers

Tablet preparations of vitamin B12, folic acid, and retinyl palmitate respectively were tested in healthy human volunteers. As reference each person was also given the same dose of active principle in form of a commercial tablet preparation (vitamin B12: Behepan®, Pharmacia; folic acid, Folacin®, Pharmacia; retinyl palmitate: Arovit®, Roche). The observed differences in blood concentration over a given period of time are expressed as percentage of the reference, which was set at 100. Thus a result above 100 for the compositions of the invention indicates an increased plasma concentration of the active principle and thus an increased pharmacological efficacy. These tests were performed with an interval of one week.

The subjects were fasting (intake of water al owed) since 10 p.m. the day before testing. On the testing day the persons arrived at the clinic at 07.00 a.m. An intravenous catheter was installed in an arm vein for sampling of blood. The tablet was taken at about 7.30 a.m. A series of blood samples were drawn as outlined in Table 3. In addition

TABLE 3
Plasma sampling pattern for vitamin B12, folic
acid, and retinyl palmitate

| | | | | | | | |
|---|---|---|---|---|---|---|---|
| Hours after dosing | 0,5 | 1 | 2 | 3 | 4 | 6 | 8 |

Compound

| | | | | | | | |
|---|---|---|---|---|---|---|---|
| Vitamin B12 | | x | x | x | x | x | x |
| Folic acid | x | x | x | x | x | x | x |
| Retinyl palmitate | | x | x | x | x | x | x |

a pre-dosing sample was taken. A standardised lurch was served after the sampling at 4 hours after administration.

The blood samples were treated and analysed in accordance with GCP and validated analytical methods provided by the Laboratory of Clinical Chemistry; Lund University Hospital, Lund, Sweden, and the Laboratory of Clinical Chemistry, Huddinge Hospital, Sweden. Plasma concentrations were plotted against time. The area under the curve obtained from the reference tablet was defined as 100, and the area under the curve (AUC) from the corresponding tablet of the invention was expressed as a percentage of the reference.

The AUC was calculated by the linear trapezoidal rule to the last blood concentration. Except for preparations no. 13, 14, 15 the concentration of the sarples taken before administration was regarded as baseline End subtracted from the concentration of sample taken after administration since no samples prior to administration were taken in the latter preparations; the plasma conc. of active principle at start was set to zero. The results are give in Tables 2 an 4-6. # Indicates the preparation number (see Table 2).

TABLE 4
Serum concentration pmol/L) off vitamin B12

| Time (h) | Ref. | #1 | #3 | #10 | Ref. | #2 | #5 |
|---|---|---|---|---|---|---|---|
| 0 | 310 | 379 | 300 | 305 | 274 | 258 | 281 |
| 1 | 409 | 358 | 1130 | 323 | 373 | 319 | 387 |
| 2 | 388 | 375 | 861 | 337 | 376 | 290 | 363 |
| 3 | 420 | 404 | 893 | 346 | 385 | 353 | 369 |
| 4 | 421 | 400 | 807 | 352 | 392 | 354 | 397 |
| 6 | 413 | 457 | 787 | 361 | 375 | 330 | 384 |
| 8 | 431 | 452 | 710 | 370 | 357 | 355 | 397 |
| % Ref. | | 33 | 529 | 43 | | 74 | 100 |
| Ref. | #4 | #7 | Ref. | #6 | Ref. * | #8* | #9* |
| 183 | 177 | 169 | 233 | 218 | 279 | 319 | 293 |
| 239 | 426 | 376 | 262 | 574 | 361 | 382 | 301 |
| 295 | 375 | 316 | 303 | 473 | 445 | 475 | 290 |
| 293 | 367 | 341 | 317 | 444 | 446 | 496 | 297 |
| 311 | 380 | 302 | 317 | 432 | 437 | 495 | 298 |
| 285 | 366 | 313 | 311 | 412 | 431 | 521 | 309 |
| 288 | 343 | 311 | 254 | 397 | 443 | 465 | 315 |
| % Ref. | 191 | 148 | | 355 | | 108 | 6 |

| Ref. | #11 | Ref. | #12 |
|---|---|---|---|
| 233 | 236 | 271 | 306 |
| 354 | 309 | 286 | 284 |
| 330 | 350 | 322 | 274 |
| 316 | 310 | 313 | 280 |
| 316 | 314 | 324 | 252 |
| 313 | 241 | 329 | 348 |
| 323 | 330 | 337 | 301 |
| % Ref. | 71 | | 0 |

TABLE 5
Serum concentration (nmol/L) of folic acid

| Time (h) | Ref. | #13 | #15 | Ref. | #14 |
|---|---|---|---|---|---|
| 0,5 | 25 | 132 | 48 | 9 | 113 |
| 1 | 50 | 399 | 193 | 10 | 193 |
| 2 | 147 | 504 | 204 | 162 | 208 |
| 4 | 525 | 231 | 170 | 273 | 252 |
| 8 | 120 | 63 | 95 | 86 | 97 |
| % Ref. | | 93 | 56 | | 117 |

| Time (h) + A55 | Ref. | #16 | #17 |
|---|---|---|---|
| 0 | 14,8 | 25 | 23,7 |
| 0,5 | 24,8 | 96 | 21,7 |
| 1 | 301 | 429 | 20,7 |
| 2 | 679 | 477 | 26,2 |
| 3 | 453 | 337 | 27,2 |
| 4 | 338 | 318 | 29,8 |
| 6 | 216 | 152 | 27,6 |
| 8 | 117 | 100 | 26 |
| % Ref. | | 81 | 1 |

TABLE 6
Serum concentration (micromol/L) of retinyl
palmitate

| Time (h) | Ref. | #18 | #19 |
|---|---|---|---|
| 0 | 0,02 | 0,04 | 0,03 |
| 1 | 0,03 | 0,07 | 0,03 |
| 2 | 0,04 | 0,48 | 0,04 |
| 3 | 0,38 | 1,19 | 0,05 |
| 4 | 0,79 | 0,92 | 0,05 |
| 6 | 1,83 | 1,52 | 0,13 |
| 8 | 0,53 | 0,64 | 0,12 |
| % Ref. | | 115 | 6 |

### EXAMPLE 5

Test of tablet preparations with desmopressin (anti-diuretic) in healthy human volunteers. This tablet preparation was tested by means of measuring the amount of urine produced over a given period of time according to procedures described in the literature (Hans Vilhardt and Stefan Lundin, Gen. Pharmac. 17 (1986) 481-483). The healthy male volunteers were fasting since 10.00 p.m. the day preceding the test. On the following morning the subject drank an amount of tap water corresponding to 1.5% of his body weight. Then the urine was collected every 15 minutes. The collected volume was measured and an equal volume of tap water was ingested immediately thereafter. The tablet was taken when the collected volume of urine per period of 15 min exceeded 150 ml. A light breakfast was given one hour after administration of desmopressin, and a light lunch 3 hours later. The liquids consumed to these meals were included in the ingested volumes replacing the collected urine.

The result of the test is expressed as percentage of the accumulated urine production in the tablet of this invention compared to half of the commercial reference tablet containing 100 μg of desmopressin (Minirin®, Ferring) over a period of 11 hours starting 30 min after administration.

The desmopressin composition according to the invention (Preparation 17) increased the anti-diuretic effect of desmopressin 3.5 times in terms of volume of urine produced over a period of 11.5 hours after administration (see, Tables 2 and 7).

### EXAMPLE 6

### Preparation of a Carbohydrate Coated Continuous Lipid Phase Tablet

Vitamin B12 tablets (EXAMPLE 2; 60 g) were fed to a coating cylinder. Simultaneously a powderous mixture of 68% acacia gum, 20% lactose and 12% dextrose (3% by weight of the tablets) was introduced into the cylinder. The mixture was rotated at 30 rpm for 3 hrs at 18° C. The tablets with a smooth surface obtained can be further coated by traditional pharmaceutical coating methods, such as by fluidised bed coating (see, for instance: S C Porter and C H Bruno, Coating of Pharmaceutical Solid-Dosage Forms, in: Pharmaceutical Dosage Forms, H A Lieberman et al., Eds., 2^{nd} Ed. Vol. 3, p. 77-160, Marcel Dekker, New York and Basel 1990, and literature cited therein).

TABLE 7
Urine collected after administration of desmopressin

| | Collected urine (ml) | |
|---|---|---|
| Min after administration | Preparation 17 | Reference |
| 30 | 0 | 42 |
| 45 | 0 | 0 |
| 60 | 0 | 0 |
| 75 | 0 | 24 |
| 90 | 0 | 0 |
| 105 | 0 | 0 |
| 120 | 0 | 0 |
| 150 | 0 | 0 |
| 180 | 0 | 50 |
| 210 | 0 | 42 |
| 240 | 140 | 48 |
| 270 | 46 | 60 |
| 300 | 34 | 66 |
| 330 | 32 | 84 |
| 360 | 30 | 120 |
| 390 | 18 | 120 |
| 420 | 20 | 158 |
| 450 | 44 | 206 |
| 480 | 70 | 208 |
| 510 | 40 | 216 |
| 555 | 28 | 322 |
| 600 | 42 | 438 |
| 645 | 98 | 448 |
| 690 | 236 | 432 |
| Accumulated volume | 878 | 3084 |

### EXAMPLE 7

### Preparation of a Continuous Lipid Phase Tablet Containing 1.8 mg of Porcine Insulin (Method B)

Materials, by weight:
Non-polar lipids (medium chain monoglycerides; MCMG) 180 parts;
Non-polar lipids (fractionated triglycerides; palmkernel stearin), 450 parts;
Polar liquad material (galactolipids; CPL-Galactolipid™), 240 parts;
Insulin, 0.8 parts;
4% Aqueous sodium bicarbonate, 28.2 parts.

Porcine insulin (Sigma, no. 15523) was dissolved in the sodium bicarbonate solution at 60° C. The monoglyceride was added and the mixture was stirred until a clear liquid had formed. The galactolipids and the palmkernel stearin were subsequently added stepwise at the same temperature. Stirring was continued until clear liquids had formed. On cooling the liquid corresponding to the tablet composition solidified; m.p. 33° C. Aliquots (500 mg) of the molten composition were cast in a mould covered with hydrogenated triglyceride (Akofine NF™) powder. The mould was cooled in a freezer. Upon solidification the solid tablets were recovered by hand.

### EXAMPLE 8

### Useful Commercially Available Synthetic Lipid Materials (Examples)

Mono- and diglyceride acetates; mono- and diglyceride citrates; mono- and diglyceride lactates; polyglycerol esters of fatty acids; propyleneglycol esters of fatty acids; sorbitane esters of fatty acids; Sodium and calcium stearoyl lactates; diacetyl tartaric acid esters of mono- and diglycerides; diglycerol esters of fatty acids.

### EXAMPLE 9

### Useful Commercially Available Food Supplement and Other Supplementary Materials for Incorporation into a Tablet of the Invention (Examples)

Amino acids, vitamins and other food supplement agents, in particular lecithin, linseed oil, melatonin, mono-octanoin, peptides, in particular di- to decapeptides, biotin, carnitine, cystine, methionine, isoleucine, leucine, ornithine, lysine acetate, folic acid, vitamin D, cholecalciferol, Vitamin E.

### EXAMPLE 10

### Gentamycin Sulphate Compositions

The following gentamycin sulphate compositions of the invention (“Gentamycin 2”, “Gentamycin 3”, “Gentamycin 4”) were prepared (Table 8).

TABLE 8
Gentamycin sulphate compositions

| Batch # | Gentamycin sulphate | Composition |
|---|---|---|
| W 21212-N1 | Gentamycin sulphate | Gentamycin sulphate 100% |
| “Gentamycin 1” | batch no. 070K1038; | |
| | Experimental batch | |
| | size: 120 mg | |
| W 20920-N3 | Gentamycin sulphate | Gentamycin sulphate 50 mg = |
| “Gentamycin 2” | batch no. 070K1038; | 1.05%; |
| | Experimental batch | H₂O 0.5 g = 10.5%; |
| | size: 2 × 4.75 g | Lyso-PC 0.5 g = 10.5%; |
| | | CPL-GL 1.05 g = 22.1%; |
| | | MCMG 1.15 g = 24.2%; |
| | | PK stearin 1.5 g = 31.6% |
| W 20920-N2 | Gentamycin sulphate | Gentamycin sulphate 50 mg = |
| “Gentamycin 3” | batch no. 070K1038; | 1.05%; |
| | | H₂O 0.5 g = 10.5%; |
| | Experimental batch | CPL-GL 1.55 g = 32.6%; |
| | size: 2 × 4.75 g | MCMG 1.15 g = 24.2%; |
| | | P stearin 1.5 g = 31.6% |
| W 21106-N2 | Gentamycin sulphate | Gentamycin sulphate |
| “Gentamycin 4” | batch no. 070K1038; | 120 mg = 3%; |
| | Experimental batch | H₂O 0.4 g = 10%; |
| | size: 4.0 g | HGL 1.24 g = 31%; |
| | | MCMG 0.92 g = 23%; |
| | | PK stearin 1.32 g = 33% |

Abbreviations in Table 8: Lyso-PC: lysophosphatidylcholine; HGL: partially hydrolysed galactolipid (Example 12); MCMG: medium chain monoacylglycerol; CPL-GL: CPL galactolipid; PK stearin: palm kernel oil stearin; P stearin: palm oil stearin.

### EXAMPLE 11

### Vancomycin Hydrochloride Compositions

The following vancomycin hydrochloride compositions of the invention (Table 9) were prepared by pouring aliquots of the liquid compositions at 50° C. into hard gelatin capsules and allowing them to cool and solidify in place.

TABLE 9
Vancomycin hydrochloride compositions

| | Batch | | |
|---|---|---|---|
| | size | | |
| Batch # | (g) | Composition | Observations |
| W 21029-N1 | 1.0 | Vancomycin hydrochloride: | |
| “Vancomycin 1” | | 20 mg = 2%; H₂O: 0.15 g = | |
| | | 15 %; HGL: 0.16 g = 16%; | |
| | | CPL-GL: 0.14 g = 14%; | |
| | | MCMG: 0.22 g = 22%; | |
| | | PK stearin: 0.31 g = 31% | |
| W 21107-N1 | 1.0 | Vancomycin hydrochloride: | Substantial |
| “Vancomycin 2” | | 20 mg = 2%; H₂O: 0.15 g = | improvement over |
| | | 15%; CPL-GL: 0.31 g = | “Vancomycin 1” |
| | | 31%; MCMG: 0.23 g = | |
| | | 23%; cholesterol 0.1 g = | |
| | | 10%; PK stearin: 0.19 g = | |
| | | 19% | |
| W 21209-N3 | 6.0 | Vancomycin hydrochloride: | Ca. 85% of water |
| “Vancomycin 3” | | 120 mg = 2%; H₂O: 0.9 g = | can be removed |
| | | 15%; CPL-GL: 1.86 g = | by evaporation |
| | | 31%; MCMG: 1.38 g = | at 60° C.; |
| | | 23%; cholesterol 0.6 g = | improvement over |
| | | 10%; PK stearin: 1.14 g = | “Vancomycin 2” |
| | | 19% | |

For abbreviations, see Table 8

### EXAMPLE 12

### Preparation of Partially Hydrolysed Galactolipid (HGL)

Galactolipid (40 g) was dissolved in MeOH (2.0 L) assisted by ultrasound. Aqueous NH₃ (25%; 10 ml) was added. The mixture was shaken at room temperature for 23 hrs; a yellowish green colour and a small amount of a lightly coloured precipitate had formed. The solution was evaporated on a rotary evaporator under reduced pressure. 400 ml of acetone was added to extract free fatty acids. After repeated evaporation at 60° C. and standing over night the supernatant was decanted and the residue evaporated and freeze dried after addition of water (300 ml). 31.7 g of a gel containing about 12% of DGMG (digalactosyl-monoacylglycerol), less than 1% of fatty acid methyl esters, and about 2% of digalactosyl-glycerol was formed. The content of DGDG (digalactosyl-diacylglycerol) thus had been reduced to about 40%.

### EXAMPLE 13

### Administration of Gentamycin

NZW rabbits were used in all experiments and all tablet/capsules were administered orally. The animals were given four, five or six tablets/capsules followed by water until they had swallowed the tablets/capsules. The animals were deprived of food for about 18 hours before dosing. Blood samples were drawn from the ear veins in sodium citrate vials before dosing and 0.5, 1, 2, 6 and, in some cases, 3 hours after dosing. The blood samples were centrifuged for 10 min at approximately 2000×g to obtain plasma for determination of gentamycin by EMIT 2000 TDM assay on a Hitachi 704 Analyzer (Table 10).

The area under the curve (AUC) was calculated by the linear trapezoidal rule to the last blood concentration. Two different doses (5 or 10 mg/kg bodyweight) were used during the experiments. For comparison of the results of the different formulations the AUC was divided by the respective dose of gentamycin. The obtained plasma concentration for pure gentamycin was set to 1. The obtained plasma concentrations for gentamycin in the three different lipid formulations were then expressed as multiple factors of increasing bioabsorption. Thus, Gentamycin 2 gave 12 times higher absorption than Gentamycin 1 due to incorporation of gentamycin in the lipid matrix.

TABLE 10
Plasma concentration of gentamycin (microgram/mL)
after oral administration to rabbits

| Time | Gentamycin 1 | Gentamycin | Gentamycin | Gentamycin |
|---|---|---|---|---|
| after | (in substance) | 2 (in lipid | 3 (in lipid | 4 (in lipid |
| adminis- | Dose 10 | matrix); dose | matrix); dose | matrix); dose |
| tration | mg/kg; | 5 mg/kg; | 5 mg/kg; | 10 mg/kg; |
| (hrs) | n = 3 | n = 4 | n = 4 | n = 3 |
| 0 | 0 | 0 | 0 | 0 |
| 0.5 | 0.01 | 0.07 | 0.09 | 0.10 |
| 1 | 0.01 | 0.22 | 0.04 | 0.07 |
| 2 | 0.02 | 0.11 | 0.07 | 0.08 |
| 3 | — | 0.06 | 0.06 | — |
| 4 | 0.01 | 0.06 | 0.05 | 0.09 |
| 6 | 0.03 | 0.09 | 0.06 | 0.08 |
| AUC | 0.09 | 0.55 | 0.34 | 0.48 |
| AUC | 0.009 | 0.11 | 0.068 | 0.048 |
| adjusted | | | | |
| to mg/kg | | | | |
| given dose | | | | |
| Correla- | 1 | 12 | 8 | 5 |
| tion | | | | |
| factor | | | | |

1-35. (canceled)

36. A solid pharmaceutical or food supplement tablet composition which has a melting point of 25° C. or higher, comprising a continuous lipid component comprising one or more glycolipids, one or more non-polar lipids, optionally one or several of water and mono- to trivalent alcohol in an amount of up to 15% by weight of the composition, and one or more agents selected from pharmacologically active agent and food supplement agent.

37. The composition of claim 36, wherein said glycolipid(s) are selected from galactolipids.

38. The composition of claim 36, consisting essentially of one or more glycolipids, one or more non-polar lipids, and one or more pharmacologically active agents or food supplement agents.

39. The composition of claim 36, consisting essentially of one or more galactolipids, one or more non-polar lipids, and one or more pharmacologically active agents or food supplement agents.

40. The composition of claim 37, wherein at least one of said galactolipid(s) is partially hydrolysed galactolipid.

41. The composition of claim 36, wherein said one or more non-polar lipids are glyceride esters of fatty acids.

42. The composition of claim 36, wherein said one or more non-polar lipids are lipids of vegetable origin.

43. The composition of claim 42, wherein said one or more non-polar lipids include triglycerides selected from palmkernel oil fractions obtained by fractionation of palmkernel oil.

44. The composition of claim 42, wherein said one or more non-polar lipids comprise C₈-C₁₀ monoglycerides and/or C₁₆-C₁₈ monoglycerides.

45. The composition of claim 36, comprising water or one or more of mono- to trivalent alcohol.

46. The composition of claim 45, wherein the monovalent alcohol is ethanol.

47. The composition of claim 45, wherein the divalent to trivalent alcohol is selected from the group consisting of 1,2-propylene glycol, low molecular weight polyethylene glycol, and glycerol.

48. The composition of claim 36, wherein said pharmacologically active agent is selected from the group consisting of analgesics, anti-inflammatory agents, antihelmintics, antiallergic agents, arrhythmic agents, antibacterial agents, anticoagulants, antidepressants, antidiabetic agents, antiepileptics, antifungal agents, antigout agents, antihistamines, antihypertensive agents, antimalarial agents, antimuscarinic agents, antimycobacterial agents, antineoplastic agents, antiprotozoal agents, antithyroid agents, antiviral agents, anxiolytic agents, beta-adrenoceptor blocking agents, cardiac inotropic agents, corticosteroids, cough suppressants, diagnostic agents, diuretics, dopaminergics, enzymes, gastro-intestinal agents, hypnotics, hypothalamic hormones, immunological agents, immunosuppresants, lipid regulating agents, mucolytics, muscle relaxants, neuroleptics, nutritional agents, opoid analgesics, parasympathomimetics, pituitary hormones, parathyroid agents, prostaglandins, sedatives, sex hormones, sympathomimetics, thyroid agents, vasodilators, vitamins, and xanthines.

49. The composition of claim 36, comprising water in an amount up to 10%.

50. The composition of claim 49, comprising up to 5% by weight of water.

51. A process for the production of a pharmaceutical or food supplement tablet composition which has a melting point of from 25° C. and higher, comprising:
mixing one or more glycolipids, with one or more non-polar lipids at a first temperature at which at least one of said components is in a liquid state,
dissolving, in the liquid continuous lipid phase obtained, one or more pharmacologically active agents,
dissolving, in the liquid continuous lipid phase obtained, one or more pharmacologically active agents,
cooling the solution of said one or more pharmacologically active agents or food supplement agents in the lipid phase to a second temperature at which it solidifies, and either
forming tablets by carrying out the cooling step with aliquots of the solution or from a bulk product obtained in the cooling step or
forming filled capsules, by carrying out the cooling step with aliquots of the solution that had been poured into said capsules.

52. The process of claim 51, wherein said first temperature is at least 25° C. and higher.

53. The process of claim 51, wherein said solution is cooled in bulk, and a powderous product is formed from said bulk product.

54. The process of claim 51, wherein said solution is fed to a nozzle and sprayed on a surface or into a cavity having a temperature below the melting point of the liquid, thereby forming a powderous product.

55. A process for the production of a pharmaceutical or food supplement tablet comprising compressing the powderous product of claim 53 into a tablet or a suppository.

56. The process of claim 55, wherein the compression employs a compression surface having an anti-adherent thereon.

57. The process of claim 56, wherein the anti-adherent is stearic acid or a salt thereof.

58. The process of claim 53, wherein the cooling is carried out by pouring an aliquot of said solution into a mould, thereby forming a tablet or suppository.

59. The process of claim 58, wherein the mould has an anti-adherent surface.

60. The process of claim 55, comprising coating said tablet or suppository with one or more powderous pharmaceutical or food supplement excipients.

61. The process of claim 59, wherein said one or more excipients are mechanically worked into the surface of the tablet so as to form a coating.

62. A pharmaceutical or food supplement tablet or suppository consisting essentially of a continuous lipid phase, optionally comprising an inert nucleus, wherein the lipid phase optionally comprises one or more of water and mono- to trivalent alcohol in an amount of up to 15% by weight of the lipid phase, the composition having a melting point of 25° C. or higher and comprising one or more polar glycolipid, components in combination with one or more non-polar lipid components, and at least one pharmacologically active agent.

63. A pharmaceutical or food supplement tablet or suppository comprising a core which has a melting point of 25° C. or higher, the core consisting of a continuous lipid phase and optionally comprising an inert nucleus, the continuous lipid phase comprising one or more glycolipid, components, one or more non-polar lipid components, wherein the lipid phase may optionally comprise one or more of water and mono- to trivalent alcohol in an amount of up to 15% by weight of the lipid phase, and one or more pharmacologically active chemical agents, further comprising a coating consisting of pharmaceutical or food supplement excipients.

64. The tablet or suppository of claim 63, wherein the coating comprises one or more subcoats comprising pharmaceutical or food supplement excipients.

65. The tablet or suppository of claim 64, wherein the coating comprises one or more subcoats comprising pharmaceutical or food supplement excipients.

66. The tablet or suppository of claim 62, wherein the one or more pharmacologically active agent is selected from the group consisting of analgesics, antiinflammatory agents, antihelmintics, antiantiallergic agents, arrhythmic agents, antibacterial agents, anticoagulants, antidepressants, antidiabetic agents, antiepileptics, antifungal agents, antigout agents, antihistamines, antihypertensive agents, antimalarial agents, antimuscarinic agents, antimycobacterial agents, antineoplastic agents, antiprotozoal agents, antithyroid agents, antiviral agents, anxiolytic agents, beta-adrenoceptor blocking agents, cardiac inotropic agents, corticosteroids, cough suppressants, diagnostic agents, diuretics, dopaminergics, enzymes, gastro-intestinal agents, hypnotics, hypothalamic hormones, immunological agents, immunosuppresants, lipid regulating agents, mucolytics, muscle relaxants, neuroleptics, nutritional agents, opoid analgesics, parasympathomimetics, pituitary hormones, parathyroid agents, prostaglandins, sedatives, sex hormones, sympathomimetics, thyroid agents, vasodilators, vitamins, and xanthines.

67. The tablet or suppository of claim 63, wherein the one or more pharmacologically active agent is selected from the group consisting of analgesics, antiinflammatory agents, antihelmintics, antiantiallergic agents, arrhythmic agents, antibacterial agents, anticoagulants, antidepressants, antidiabetic agents, antiepileptics, antifungal agents, antigout agents, antihistamines, antihypertensive agents, antimalarial agents, antimuscarinic agents, antimycobacterial agents, antineoplastic agents, antiprotozoal agents, antithyroid agents, antiviral agents, anxiolytic agents, beta-adrenoceptor blocking agents, cardiac inotropic agents, corticosteroids, cough suppressants, diagnostic agents, diuretics, dopaminergics, enzymes, gastro-intestinal agents, hypnotics, hypothalamic hormones, immunological agents, immunosuppresants, lipid regulating agents, mucolytics, muscle relaxants, neuroleptics, nutritional agents, opoid analgesics, parasympathomimetics, pituitary hormones, parathyroid agents, prostaglandins, sedatives, sex hormones, sympathomimetics, thyroid agents, vasodilators, vitamins, and xanthines.

68. The tablet of claim 62, wherein the one or more food supplement agents are selected from the group consisting of amino acids and vitamins.

69. The tablet of claim 63, wherein the one or more food supplement agents are selected from the group consisting of amino acids and vitamins.

70. A method of treating or preventing a condition comprising administration of a pharmacologically effective dose of an agent according to claim 66.

71. A method of treating or preventing a condition comprising administration of a pharmacologically effective dose of an agent according to claim 67.
